(19) United States
(12) Patent Application Publication
(10) Pub. No.: US 2014/0214079 A1
(43) Pub. Date: Jul. 31, 2014

(54) Percutaneous hernia repair

(71) Applicants: Richard C. Ewers, Fullerton, CA (US); Eugene G. Chen, Carlsbad, CA (US)

(72) Inventors: Richard C. Ewers, Fullerton, CA (US); Eugene G. Chen, Carlsbad, CA (US)

(21) Appl. No.: 13/815,110

(22) Filed: Jan. 31, 2013

(51) Int. Cl. A61B 17/04

(52) U.S. Cl. CPC A61B 17/0401
USPC 606/232

(57) ABSTRACT

Methods for percutaneous hernia repair may include inserting a needle end of an anchor tool through a first tissue edge on a first side of a hernia defect opening. A first anchor is deployed with a first suture attached to the first anchor and running back through or along the anchor tool and outside of the patient's body. The needle end of the anchor tool is withdrawn from the first tissue edge. The first suture is separated from the anchor tool. The needle end of an anchor tool is inserted through a second tissue edge and a second anchor is deployed, with a second suture attached to the second anchor and running back through or along the anchor tool and outside of the patient's body. The sutures are tensioned and cinched or knotted.

This application claims priority to U.S. Provisional Application No. 61/593,428 filed Feb. 1, 2012, and incorporated herein by reference.

### BACKGROUND OF THE INVENTION

A hernia is a defect in the abdominal wall where the inside layers of the abdominal muscle have weakened. The defect is typically a bulge or tear in the abdominal wall. Hernias may be surgically repaired, typically by suturing and/or placing a mesh patch over the defect. Hernia repair surgery may be performed as open surgery through a single large incision through the abdomen, or laparoscopically via three small puncture incisions. Laparoscopic hernia surgery generally is less pain and faster recovery than open repair surgery, but may also be technically more complicated, and have a higher risk for serious complications, compared to open repair surgery. Regardless of the type of surgery used, closing the defect in the abdominal wall generally places the suture or stitching under substantial tension. This can result in the suture pulling through or out of the abdominal tissue, leading to a hernia recurrence or other complications. Accordingly, there is a need for improved devices and methods for hernia repair.

### BRIEF DESCRIPTION OF THE DRAWINGS

FIG. 1 is a schematic diagram of a hernia defect.

FIG. 2 is a side view of a scope usable in repairing the defect shown in FIG. 1.

FIG. 3 is a schematic diagram showing the scope of FIG. 2 inserted percutaneously through the abdomen and through the defect.

FIG. 4 is an enlarged section view of the scope shown in FIGS. 2 and 3.

FIG. 5 is a side view of an alternative scope.

FIG. 6 is an end view of the scope shown in FIG. 5, in a delivery position.

FIG. 7 is an end view of the scope shown in FIGS. 5 and 6, in an operating position.

FIGS. 8-15 are schematic diagrams of a sequence of steps of a percutaneous hernia repair method.

FIGS. 16-19 are schematic diagrams of alternative stitch patterns that may be used in performing the method shown in FIGS. 8-15.

FIG. 20 is a side view of anchor deployment catheter than may be used in performing the method shown in FIGS. 8-15.

FIG. 21 is an enlarged section view of the distal end of the catheter shown in FIG. 20.

FIG. 22 is an enlarged perspective view of one of the anchors shown in FIG. 21.

FIG. 23 is an enlarged section view of the anchor shown in FIG. 22.

FIG. 24 is schematic diagram of the proximal end of the catheter shown in FIG. 21.

FIG. 25 is an enlarged side view of the cinch shown in FIG. 12.

FIG. 26 is schematic diagram of a surgical tool for placing the cinch shown in FIGS. 12 and 25.

FIGS. 27-29 are schematic diagrams illustrating use of the tool shown in FIG. 26.

FIG. 30 is a section view of the cinch shown in FIG. 12.

FIGS. 31A-31F are views of alternative cinch designs.

FIG. 32A is schematic diagram of a modification of the surgical tool shown in FIG. 26.

FIG. 32B is schematic diagram of a surgical tool of FIG. 32A in use.

FIGS. 33-36 are schematic diagrams of an alternative surgical method.

FIGS. 37-42 are diagrams showing steps of another alternative surgical method.

FIGS. 43-44 are diagrams showing another alternative method using a spring clip deployed from a needle delivery tube.

### DETAILED DESCRIPTION OF THE DRAWINGS

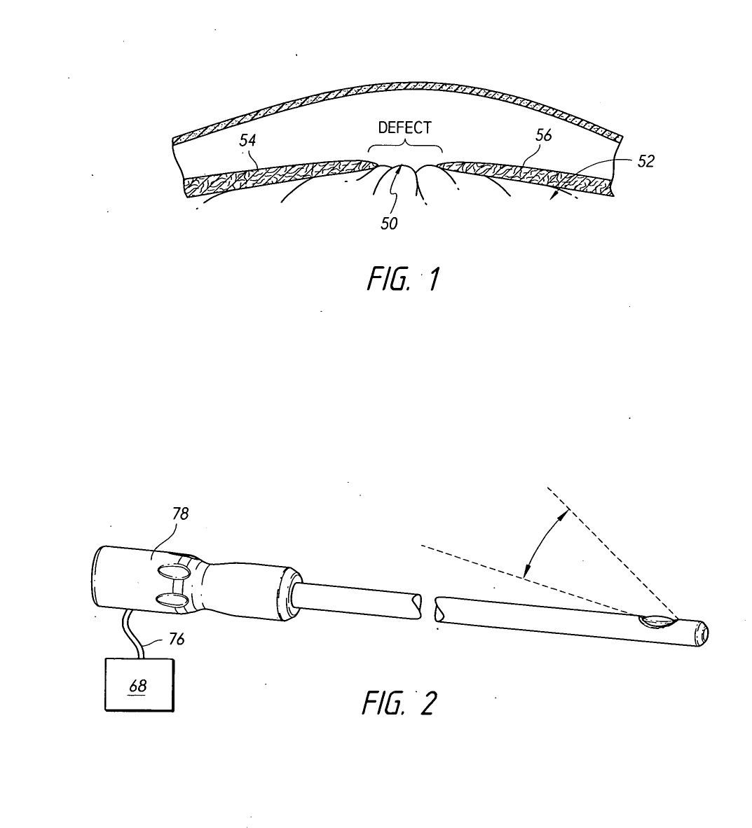

Turning in detail to the drawings, in FIG. 1 a hernia is shown as a defect 50, such as a hole or tear, in the abdominal wall 52, leaving two free sides or edges of tissue 54 and 56. As described here, the defect 50 is repaired percutaneously, meaning via surgical instruments inserted through the skin 58 of abdomen overlying the defect. In general, in the present percutaneous methods, the instruments have a small diameter, typically less than 5, 4, 3 or 2 mm in diameter. The instruments are also generally inserted through the skin, and not through any intermediate device such as a trocar as used in laparoscopic surgery (although laparoscopic techniques including use of trocars may be used in combination with the present percutaneous methods).

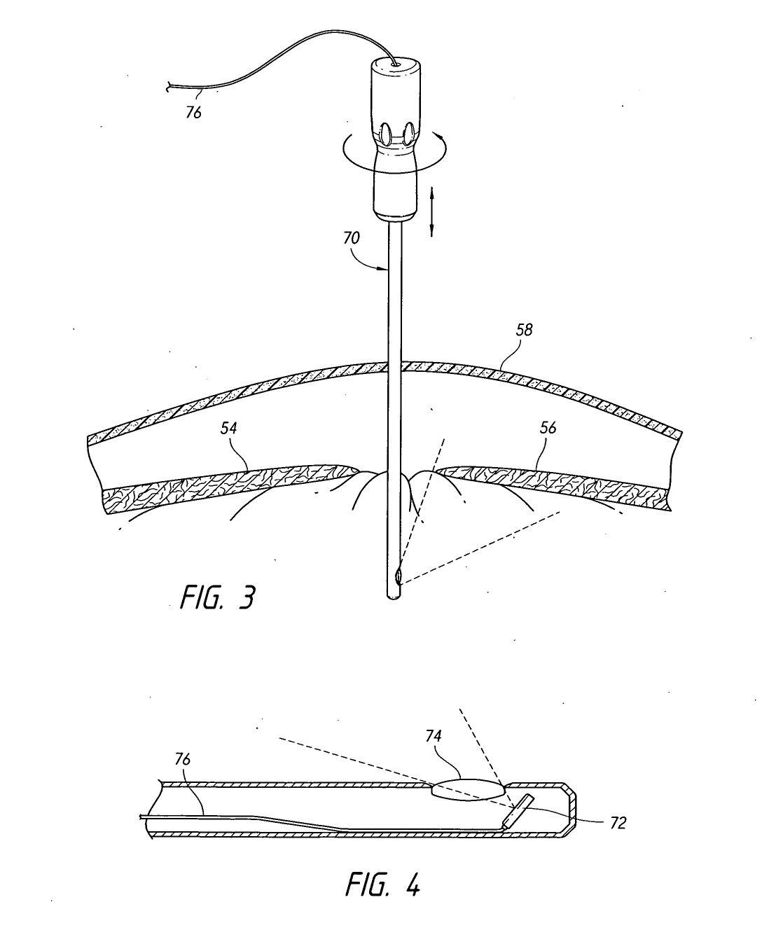

FIGS. 2-7 show devices and methods for viewing the defect 50. As shown in FIGS. 3 and 4 a scope 70 has a digital camera 72 set a reverse angle, and connected to a display 68 via a transmission cable 76. A lens 74 is positioned on the scope 70 proximal of the camera 72. The scope 70 may be inserted percutaneously through the skin 58 and through the defect 50, providing a view of the back or underside of the defect, as shown in FIG. 3, and referred to here as a reverse view.

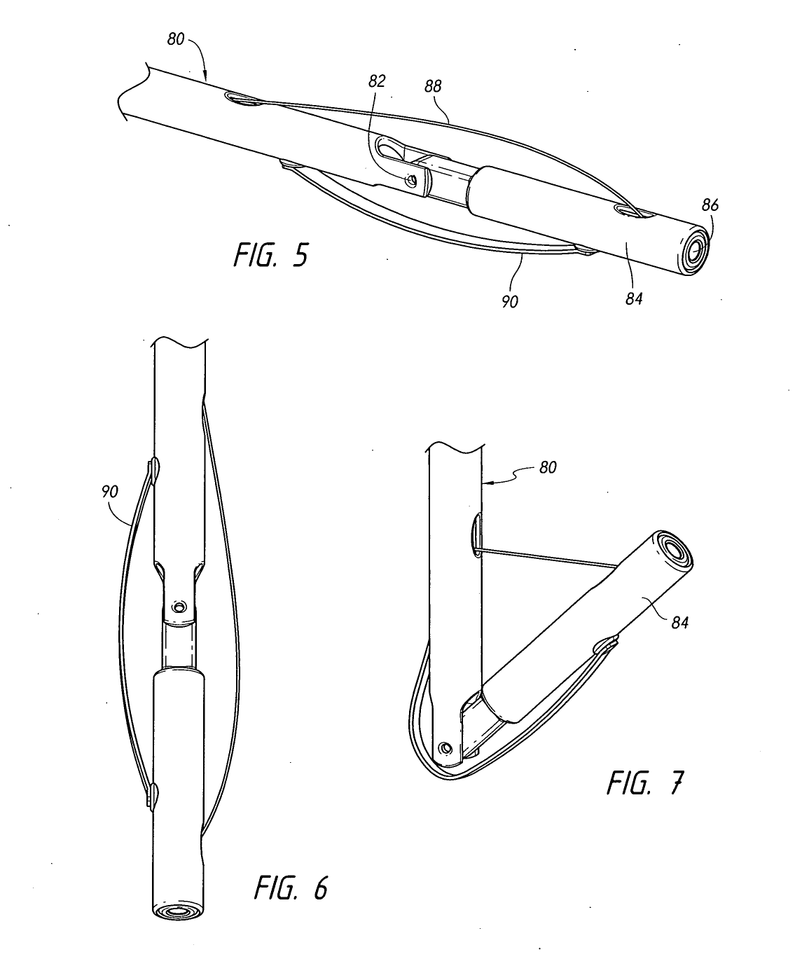

FIGS. 5-7 show an alternative scope having an end segment 84 attached to the body of the scope at a hinge joint 82. A camera 86 in on the distal end of the end segment 84. When it the scope is moved into position, the end segment is straight and aligned with the body of the scope. To provide reverse view, a pull wire 88 is pulled on the handle 78 of the scope 80. This causes the end segment to pivot over into the position shown in FIG. 7, providing a reverse view of the defect 50. Releasing tension on the pull wire 88 allows a spring 90 to return the end segment back to the original straight position, to allow the scope to be removed, or to view other areas within the abdominal cavity. If the operating surgeon already has a laparoscope placed into the abdominal cavity, then the present methods may be performed using the laparoscope, and the percutaneous scopes 70 or 80 are not needed.

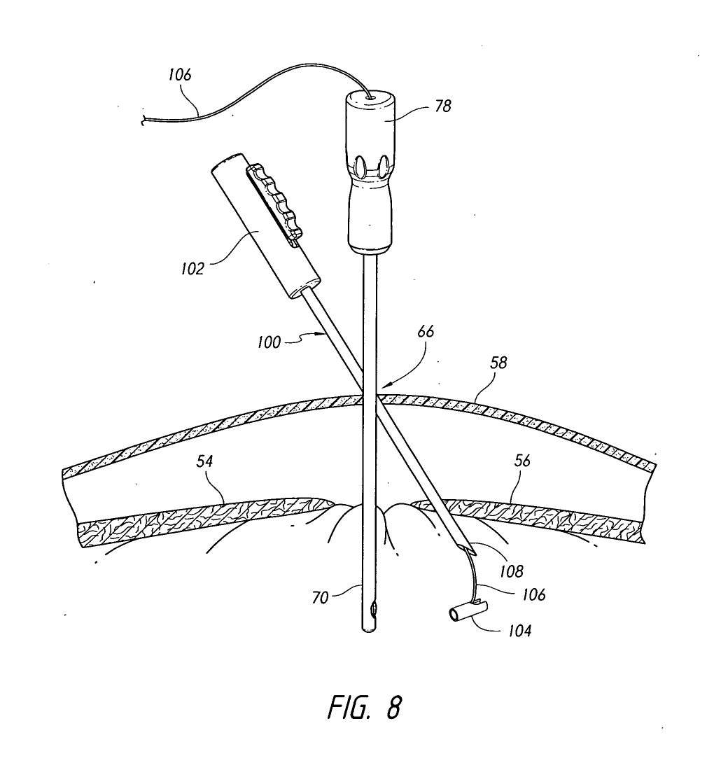

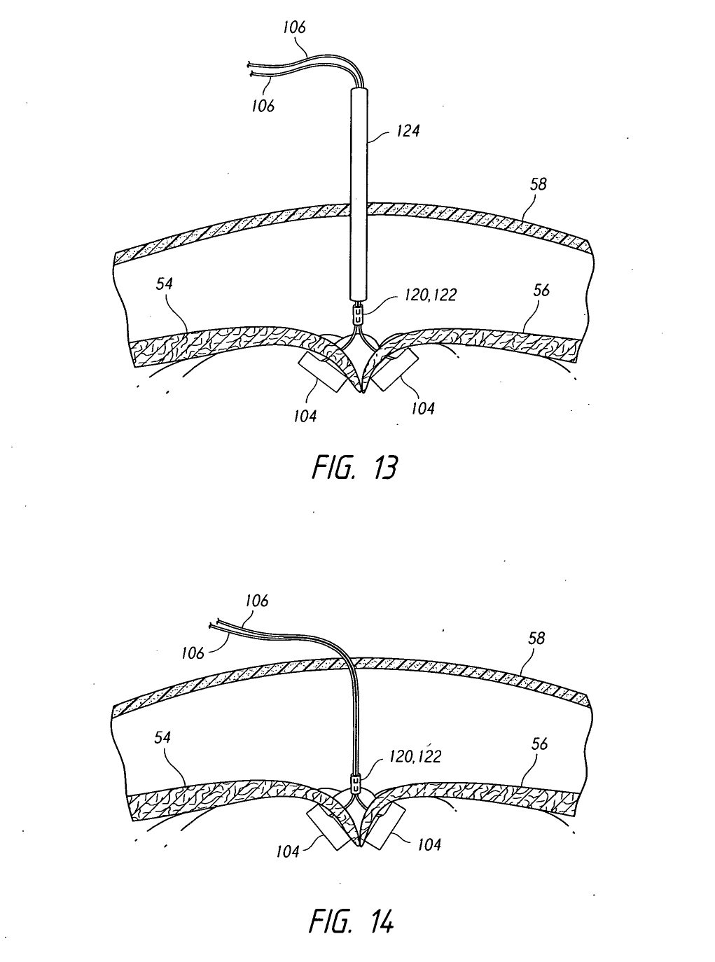

FIGS. 8-15 show a sequence of steps of a percutaneous hernia repair method. The defect 50 is shown as tear in the abdominal wall. However, the present devices and methods are also usable for performing various other types of procedures, such as repairing ventral wall defects, incisional hernias, and other types of hernias. As shown in FIG. 8, an anchor tool 100 has a handle 102 and a needle end 108. An anchor 104 near the needle end 108 is attached to suture 106 which runs back up through the tool, or outside of the tool, and out of the handle or other position on the tool that remains outside of the patient's body. The anchor tool is percutaneously introduced into the abdominal cavity by piercing through the skin 58 using the sharp needle end 108, or by inserting the needle end through an existing percutaneous puncture opening 66. The abdominal cavity is typically insufflated with gas under pressure, to create space for viewing the surgical site.

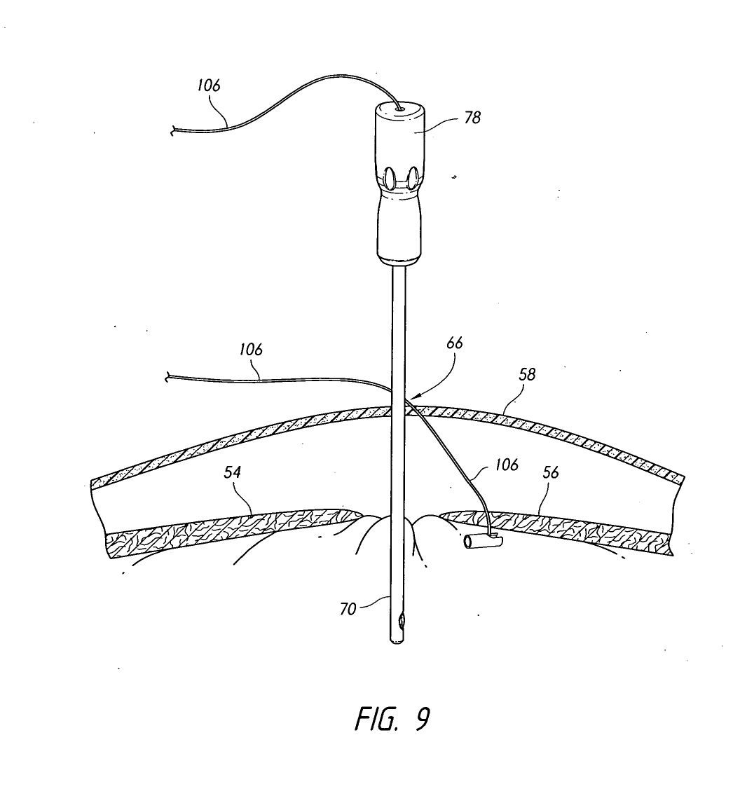

Using the scope 70 or other viewing device, the needle end 108 is passed through the first tissue edge 56. The tissue edge 56 is generally taut, allowing the needle end 108 to pierce through it, without using any other grasping or tensioning tool, although another assisting tool may optionally be used. The surgeon then uses the tool 100 to deploy an anchor on the underside of the tissue edge. As shown in FIG. 9, the tool 100 is then withdrawn out of the body, with the suture 106 leading out of the body via the percutaneous opening 66 in the skin 58. The suture 106 is then separated from the tool 100. This may be achieved by pulling the tool back until the entire length of the suture is pulled through the tool. Alternatively, the tool 100 may have suture release slot which allows the suture to separated from the tool 100 when appropriately positioned.

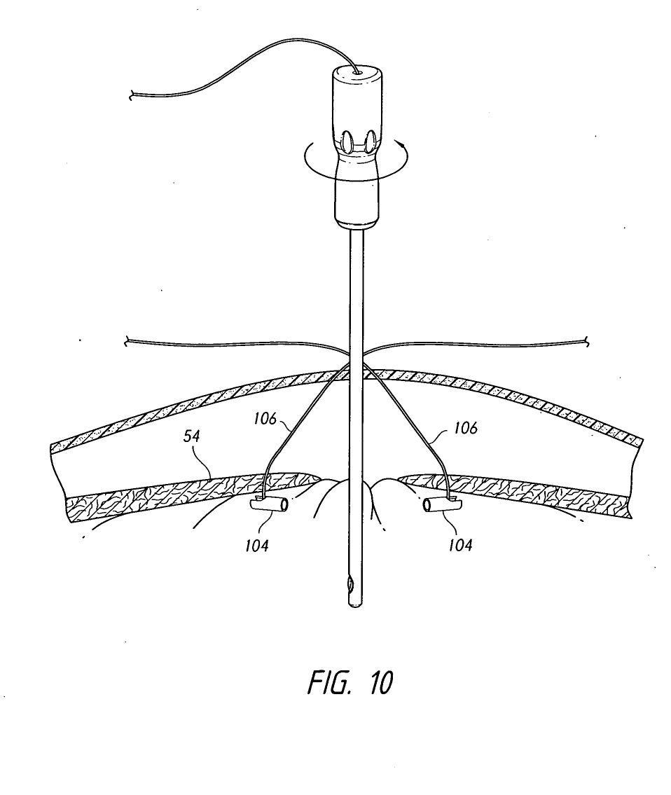

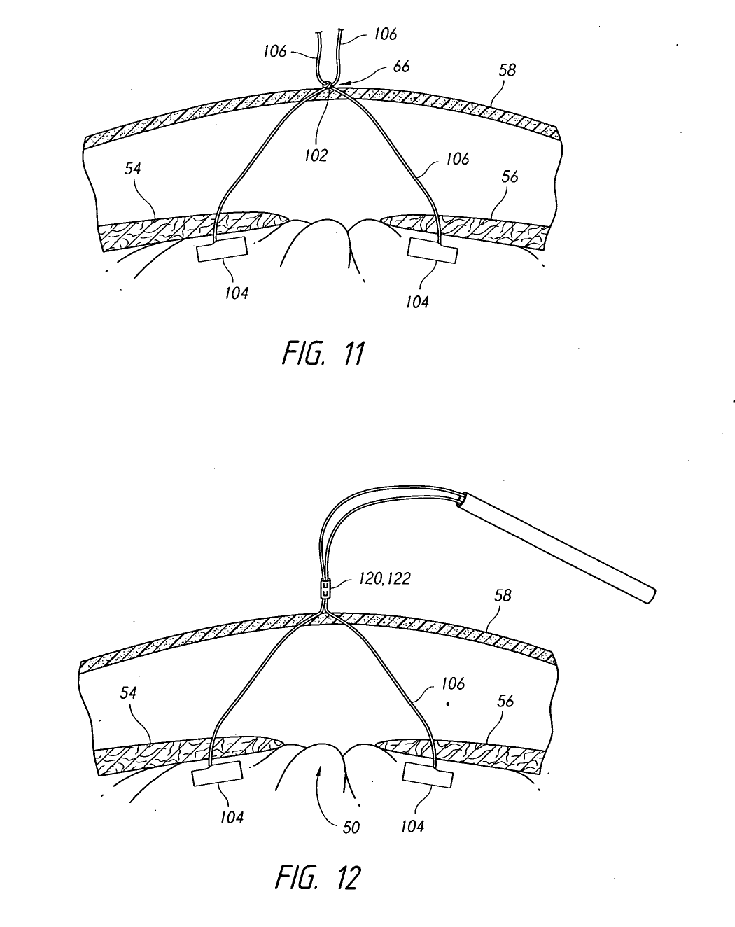

Referring to FIG. 10, the step of FIG. 9 is then repeated on the second tissue edge 54. The surgical site is then as shown in FIG. 11, with anchors 104 deployed both tissue edges, and with suture running from each anchor 104 out through the percutaneous opening 66. The scope 70 may be left in place for performing the next step, or it may be removed.

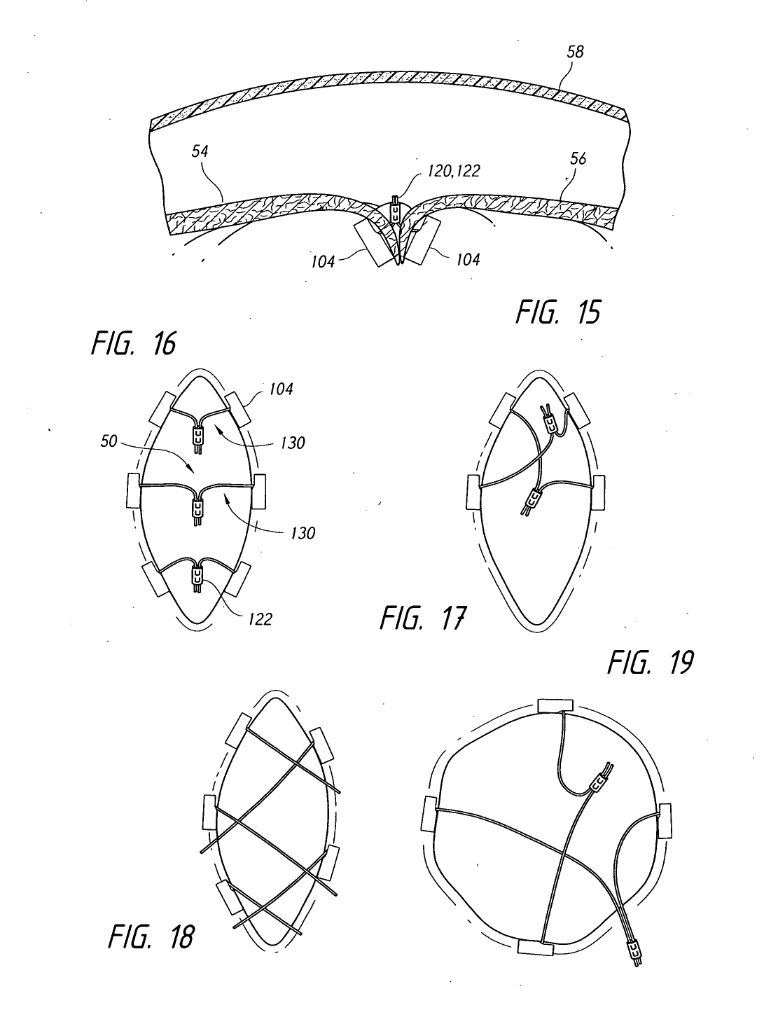

As shown in FIG. 11, the sutures 104 may be tied with a sliding knot 120, or they may be passed through a cinch 122. Referring to FIGS. 12-14, the knot or cinch is then advanced through the opening 66 using a knot pusher 124 or other tool. The distal end of the pusher 124 is pushed through the opening 66, with the knot 120 or cinch 122 sliding on the sutures ahead of the tool 124. The sutures 106 are tensioned as the knot or cinch is advanced. The distal end of the tube 124 pushes the knot or cinch forward until it is adjacent to, or in contact with, the tissue edges 54 and 56, as shown in FIG. 14. This pulls the tissue edges 54 and 56 towards each other, closing the defect 50. The pushing tool 124 may have a blade for cutting the sutures after the knot or cinch is in its final position shown in FIG. 15.

Turning to FIGS. 16-19, the steps described above may be repeated to apply a line of stitches 130 in a row, or in a pattern. A “stitch” here means first and second anchors connected to first and second sutures, respectively, secured together by a knot or cinch. Where multiple stitches 130 are used, they may be progressively tightened, to gradually bring the opposing tissue edges together. This approach reduces the force applied by any single stitch, but requires more than one tightening step on each stitch. In other cases, each stitch may be fully tightened in a single step. Where multiple stitches are used, the stitches 130 may run straight across the defect 50, as shown in FIG. 16. Alternatively, the stitches 130 may cross over each other in varying patterns, as shown in FIGS. 17 and 18. If the defect is more circular as in FIG. 19, rather than elliptical or elongated as in FIGS. 16-18, the stitches 130 may be placed in a radial pattern, as shown in FIG. 19.

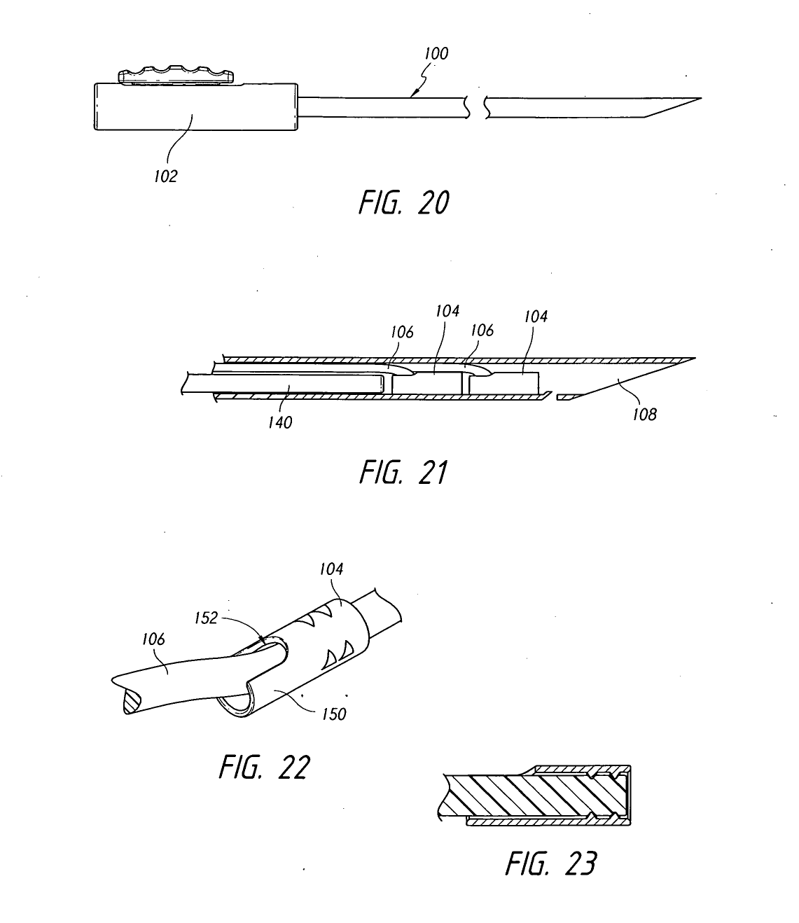

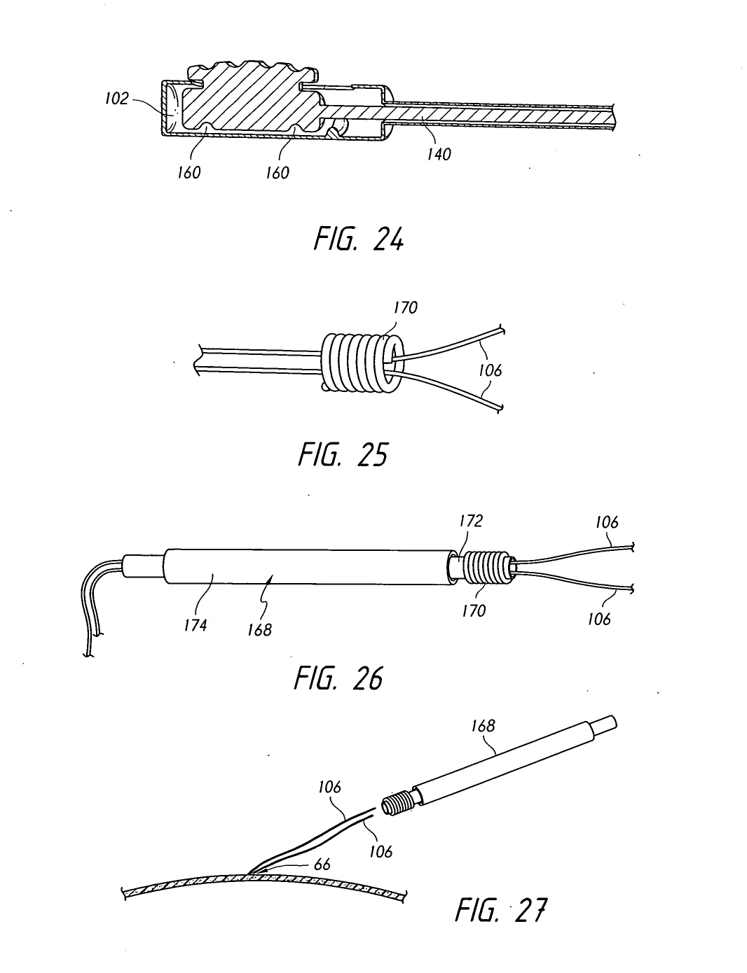

FIGS. 20-24 show designs for the anchor tool 100 and anchors 104. As shown in FIG. 21, two anchors 104 may be pre-loaded into the lumen of the tool 100, near the needle end 108. A suture or wire 106 is attached to each anchor with the sutures running back through the lumen of tool. Alternatively, the sutures can run outside of the tool and then run into the lumen of the tool near the needle end 108, to attach to the anchors. The anchors 104 may be deployed using a push rod 140 within the tool 100 to push the anchors out through the needle end 108. In the example shown in FIGS. 22 and 23, the anchor comprises a hypo tube 150 having a slot 152 at one end. The other end of the hypo tube may be crimped onto the suture 106, as shown in FIG. 23. When deployed, the suture moves out of the slot 152 into a position generally perpendicular to the hypo tube 150, forming a T-anchor. As shown in FIG. 24, the handle 102 may have indents 160 to provide a tactile feel to the surgeon indicative of first and second positions of the push rod, for deploying the anchors. Various other forms of anchors may of course also be used, such as collapsible/expandable wire frame anchors.

FIG. 25 shows a cinch 122 in the form of a wire coil 170, with the sutures 106 running through the coil 170. The coil may be Nitinol or stainless steel, or similar materials. Referring to FIG. 26, a coil cinch delivery tool 168 includes an inner tube 172 slidable relative to an outer tube 174. A coil 170 is expanded or uncoiled enough to fit over the distal end of an inner tube 172. When released, the coil 170 is held onto the end of the inner tube 172 via the internal elastic spring force of the coil material. Typically, the tool 168 may be supplied from the manufacturer with the coil 170 already in place on the inner tube 172.

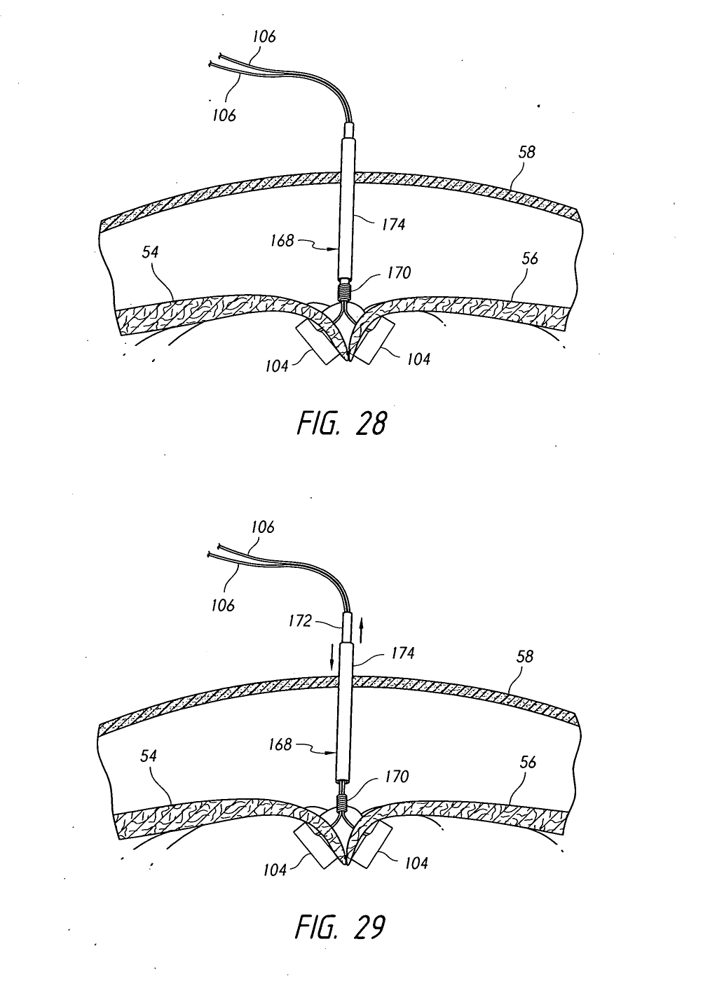

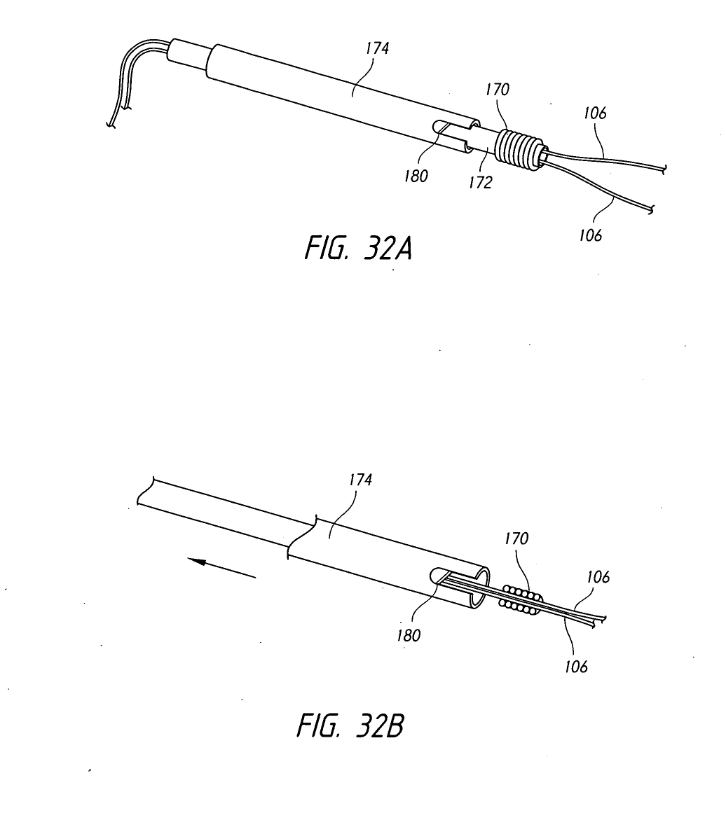

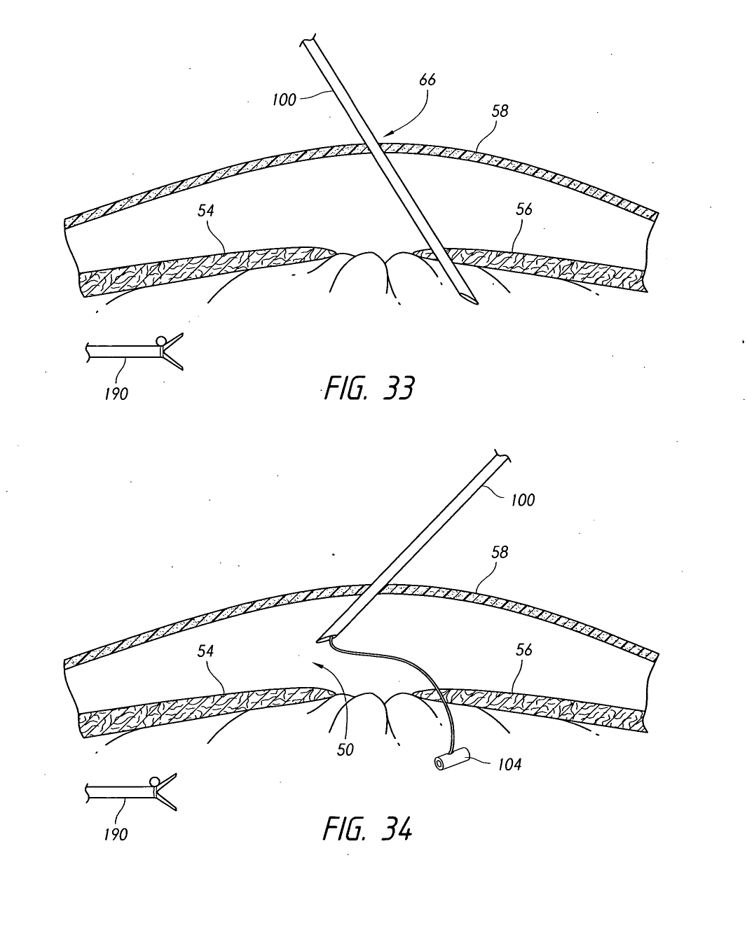

Referring to FIGS. 26-29, 32A and 32B, after the anchors 104 are placed and the back ends of the sutures brought out through the opening 66 (as shown in FIG. 9), the sutures 106 are threaded through the lumen in the inner tube 172. The tool 168 is then advanced through the opening 66, and the coil 170 moved into the defect 50, as shown in FIG. 28. With the coil 170 appropriately positioned and typically with the sutures 106 under tension, the inner tube 172 is move back relative to the outer tube 174, pushing the coil 170 off of the inner tube and onto the sutures 106. The coil squeezes onto the sutures, holding the tissue edges 54 and 56 together, as shown in FIG. 29, to close the defect 50. FIGS. 32A, 32B and 33 show operation of the tool 168, with the coil pushed off of the inner tube and clamping onto the sutures. The cutter 180 on the outer tube 174 may be used to cut the sutures.

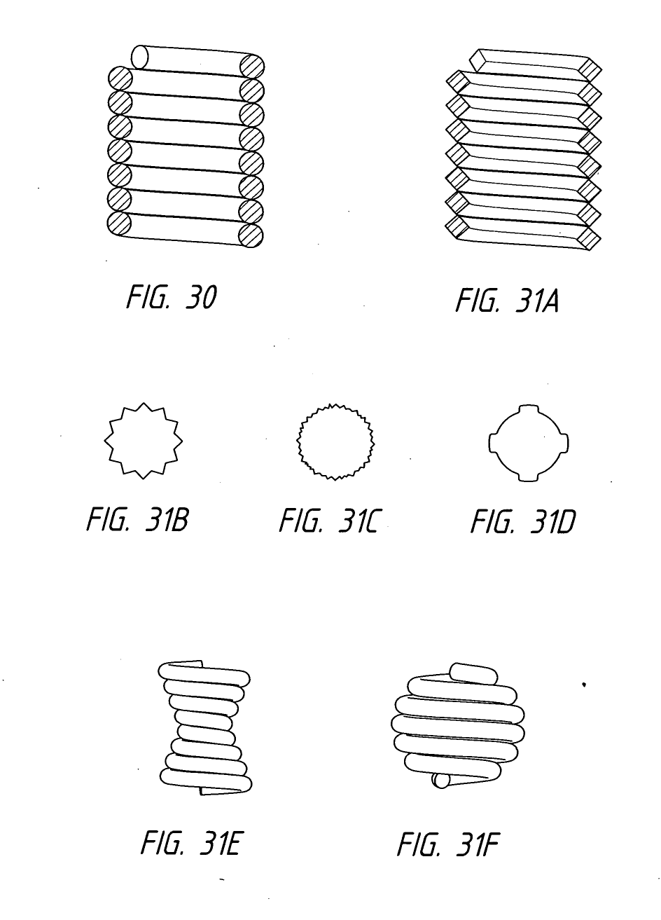

FIG. 30 shows a cross section of a coil 170 made with wire having a round cross section. FIG. 31A shows a cross section of a coil 170 made with wire having a square cross section. In this design, the edges of the square wire provide adding gripping effect on the sutures. FIG. 31B through 31D shown alternative coil wire profiles, specifically, a star wire having protruding ridges in FIG. 31B, a wire having a rough surface in FIG. 31C, and a wire having radial splines in FIG. 31D. In contrast to the cylindrical coil shown in FIG. 30, FIG. 31E shows an hour glass pinched center coil and FIG. 31F shows a football shape coil having smaller diameter ends.

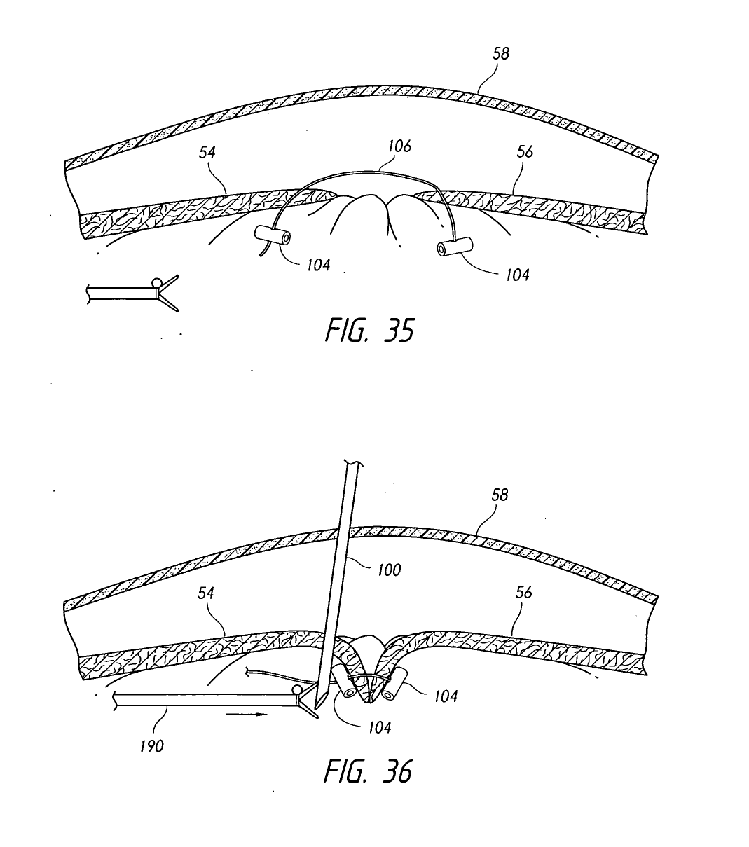

FIGS. 33-36 show an alternative method where the suture remains inside of the patient. As shown in FIGS. 33 and 34, the anchor tool 100 percutaneously pierces the first tissue edge 56 and deploys a first anchor 104. A grasping/viewing tool 190 placed percutaneously or laparoscopically provides a view of the surgical site. As shown in FIG. 35, the anchor tool 100 then pierces the second tissue edge 54 and deploys a second anchor 104 attached to the first anchor 104 via suture 106. The grasping tool 190 may assist by holding and stabilizing the tissue. The second anchor 104 either includes a one-way cinch on the suture, or a cinch separate form the anchor may be used. As shown in FIG. 36, the grasping tool 190 then pushes the cinch up against the second tissue edge 54, closing the defect 50.

In this method, as well as in the method of FIGS. 8-15, surgical mesh may also be placed over the defect, with the stitches used to close the defect and hold it closed, and/or to hold the mesh in position over the defect.

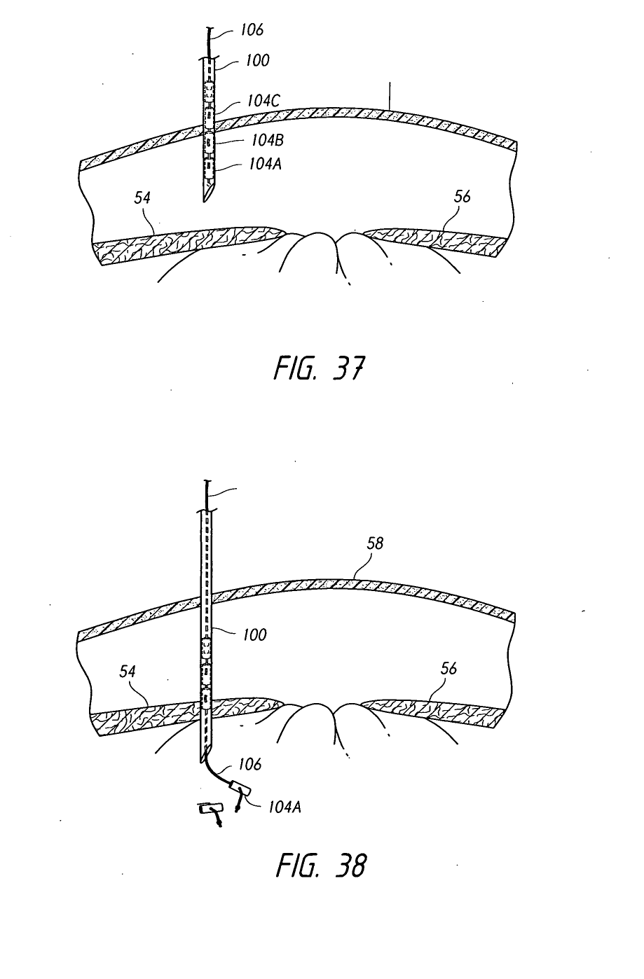

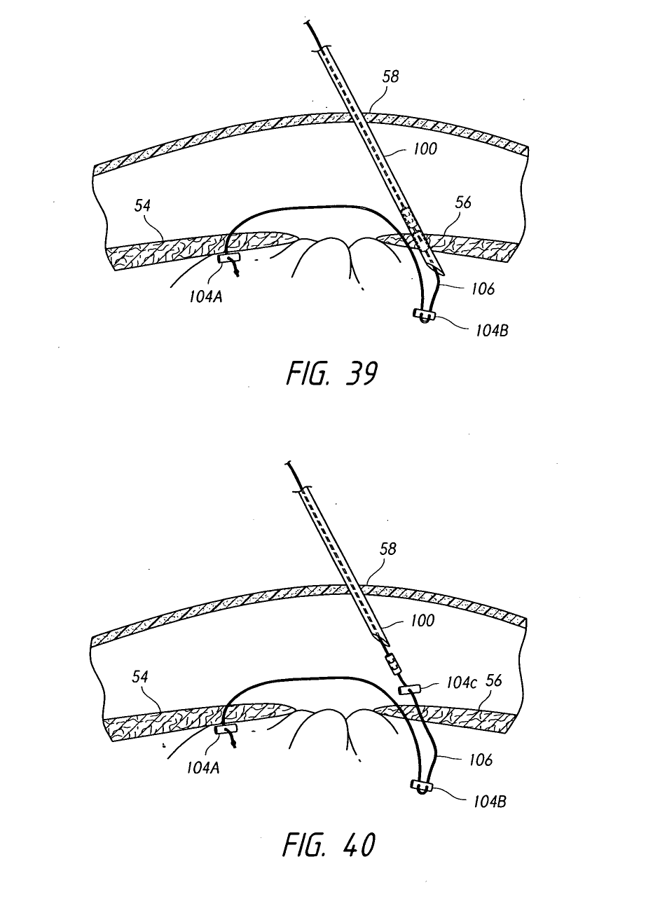

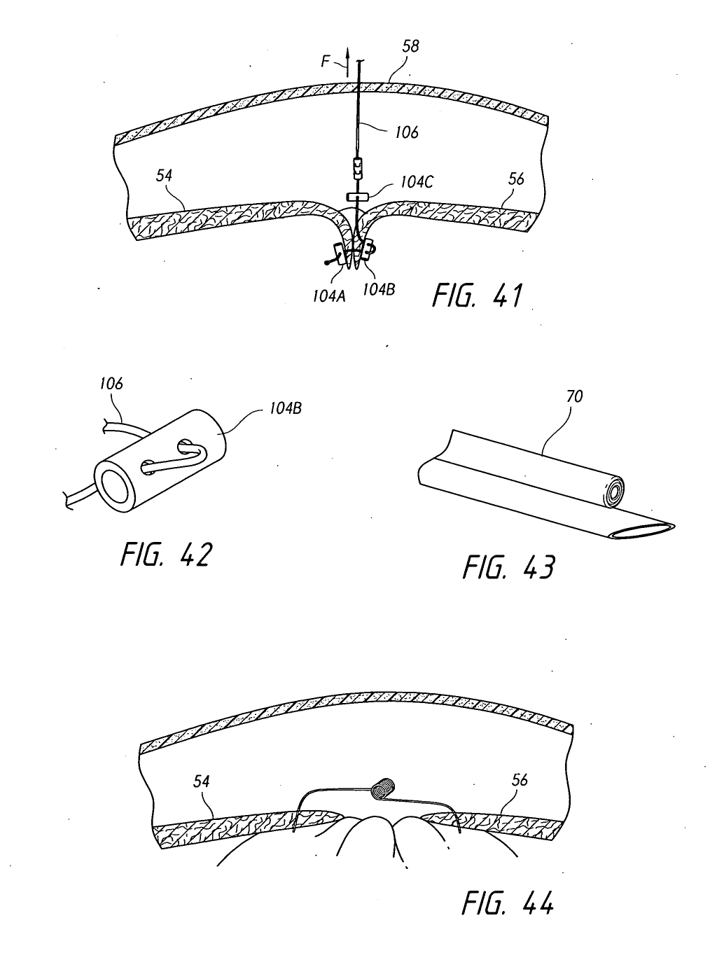

A method shown in FIGS. 37-42 is similar to the methods above except that three anchors 104A, 104B and 104C are used, instead of two anchors. The three anchors may be connected via suture and pre-loaded into the delivery tool 100, as shown in FIG. 37. The first anchor 104A is deployed through the first tissue edge 54 as in the methods above. The second anchor 104B is similarly deployed through the second tissue edge 56. However, the second anchor 104B is return anchor, as the suture 106 runs into and out of the same side of the anchor. An example of a return anchor is shown in FIG. 42. Referring to FIGS. 40 and 41, after the delivery tool 100 is pulled back through the second tissue edge, the third anchor 104C is deployed, along with a cinch on the suture or built into the third anchor. The second anchor assists in holding the tissue edges together and the third anchor and the cinch help to keep the second anchor in position.

FIGS. 43 and 44 show a method where a scope 70 is combined with a needle delivery tube 200. The delivery tube 200 is aligned over the approximated tissue edges. A spring clip 202 is deployed from the delivery tube 200. The spring clip has arms with sharp tips that pierce through the tissue edges to further hold them together.

Thus, novel devices and methods have been shown and described. Various changes and substitutions may of course be made without departing from the spirit and scope of the invention. The invention, therefore, should not be limited, except by the following claims, and their equivalents.

1. A method for percutaneous hernia repair comprising:
inserting a needle end of an anchor tool through the skin of the abdomen of the patient and through a first tissue edge on a first side of a hernia defect opening, with a handle end of the anchor tool remaining outside of the patient's body;
deploying a first anchor from the needle end of the anchor tool, with a first suture attached to the first anchor and running back through or along the anchor tool and outside of the patient's body;
withdrawing the needle end of the anchor tool from the first tissue edge;
separating the first suture from the anchor tool;
inserting the needle end of an anchor tool through the skin of the abdomen of the patient and through a second tissue edge on a second side of the hernia defect opening, with the handle end of the anchor tool remaining outside of the patient's body;
deploying a second anchor from the needle end of the anchor tool, with a second suture attached to the second anchor and running back through or along the anchor tool and outside of the patient's body;
withdrawing the needle end of the anchor tool from the second tissue edge;
separating the second suture from the anchor tool;
tensioning the first and second sutures by pulling on the first and second sutures from outside of the patient's body; and
advancing a cinch or knot along the first and second sutures towards the defect opening.

2. The method of claim 1 further comprising advancing the cinch or knot until it pulls the first tissue edge into contact with the second tissue edge.

3. The method of claim 1 further comprising inserting a viewing instrument through the skin and through the defect and viewing the first tissue edge in a reverse view.

4. The method of claim 1 further comprising threading the first and second sutures through a lumen of a pushing tool, and pushing the cinch or knot towards the anchors.

5. The method of claim 1 further comprising threading the first and second sutures through a lumen of a cinch placement tool, and placing the cinch onto the sutures at a cinch position adjacent to the first and second tissue edges.

6. A method for percutaneous hernia repair comprising:
inserting a needle end of a first anchor tool through the skin of the abdomen of the patient and through a first tissue edge on a first side of a hernia defect opening, with a handle end of the first anchor tool remaining outside of the patient's body;
deploying a first anchor from the needle end of the first anchor tool, with a first suture attached to the first anchor and running back through or along the first anchor tool and outside of the patient's body;
withdrawing the needle end of the first anchor tool from the first tissue edge;
separating the first suture from the first anchor tool;
inserting the needle end of a second anchor tool through the skin of the abdomen of the patient and through a second tissue edge on a second side of the hernia defect opening, with a handle end of the second anchor tool remaining outside of the patient's body;
deploying a second anchor from the needle end of the second anchor tool, with a second suture attached to the second anchor and running back through or along the second anchor tool and outside of the patient's body;
withdrawing the needle end of the second anchor tool from the second tissue edge;
separating the second suture from the second anchor tool;
tensioning the first and second sutures by pulling on the first and second sutures from outside of the patient's body; and
advancing a cinch or knot along the first and second sutures towards the defect opening.

7. A surgical tool comprising:
a rigid tube having an internal lumen and a needle end and a handle end;
a first anchor within the internal lumen, and a first suture attached to the first anchor and extending back through or alongside the lumen from the first anchor towards the handle end;
a second anchor within the internal lumen, and a second suture attached to the second anchor and extending back through or alongside the lumen from the second anchor towards the handle end; and
a pusher slidably positioned within the internal lumen of the rigid tube, and with the first and second anchors between the pusher and the needle end.

8. A method for percutaneous hernia repair comprising:
inserting a needle end of an anchor tool through the skin of the abdomen of the patient and through a first tissue edge on a first side of a hernia defect opening, with a handle end of the anchor tool remaining outside of the patient's body;
deploying a first anchor from the needle end of the anchor tool;
withdrawing the needle end of the anchor tool from the first tissue edge;
inserting the needle end of the anchor tool through a second tissue edge on a second side of the hernia defect opening, with the handle end of the anchor tool remaining outside of the patient's body;
deploying a second anchor from the needle end of the anchor tool, with the second anchor connected to the first anchor by suture; and
cinching the second anchor against the second tissue edge.
